(19) United States
(12) Patent Application Publication
(10) Pub. No.: US 2016/0346390 A1
(43) Pub. Date: Dec. 1, 2016

(54) METHOD OF ENHANCING RADIATION THERAPY OF CANCER

(71) Applicant: Korea Institute of Radiological & Medical Sciences, Seoul (KR)

(72) Inventors: Eun Ho Kim, Seoul (KR); Seong Hoon Yoo, Gyeonggi-do (KR); Il Sung Cho, Seoul (KR); Sung Ho Cho, Seoul (KR); Jae Ik Shin, Seoul (KR); Yong Keun Song, Seoul (KR); Won Gyun Jung, Gyeonggi-do (KR)

(21) Appl. No.: 15/166,748

(22) Filed: May 27, 2016

(30) Foreign Application Priority Data
May 29, 2015 (KR) 10-2015-0076551

(51) Int. Cl. A61K 41/00; A61K 9/00; A61K 33/24; A61K 9/14

(52) U.S. Cl. CPC A61K 41/0038; A61K 9/14; A61K 9/0019; A61K 33/24

(57) ABSTRACT

According to an aspect of the present disclosure, a method of enhancing neutron high linear-energy-transfer (LET) radiation therapy of cancer is provided, wherein the method includes administering an effective amount of gold nano-particles (GNPs) to a subject that needs neutron high LET radiation therapy of cancer, before or after radiation therapy.

### CROSS-REFERENCE TO RELATED APPLICATION

This application claims the benefit of Korean Patent Application No. 10-2015-0076551, filed on May 29, 2015, in the Korean Intellectual Property Office, the disclosure of which is incorporated herein in its entirety by reference.

### BACKGROUND

1. Field

One or more exemplary embodiments of the present disclosure relate to a method of enhancing radiation therapy of cancer, and more particularly, to a method of enhancing neutron high linear-energy-transfer (LET) radiation therapy of cancer.

2. Description of the Related Art

The methods of cancer treatment are ones such as surgery, chemical drug treatment, and radiation therapy, and in recent years, the importance of radiation therapy has been increasing more and more. In recent years, even if the surgery is difficult, it has been increasingly reported to cure tumor efficiently only with radiation therapy. In addition, methods of treating tumors using radiation are developing over time. Therefore, radiation therapy is becoming a method that can efficiently cure a tumor in the body of a patient, without arousing much pain or repulsion.

Among radiation therapies, the method of cancer treatment using LET radiation, including neutron or baryon beams, enables minimizing the radiation amount reached to normal tissues around cancer cells and providing the therapeutic amount reached to cancer cells parts only. Thus, the method using high LET radiation has been rated as far more efficient than radiation therapy using low LET radiation. High LET radiation refers to radiation with high linear-energy-transfer, and examples thereof include p-ray, α-ray, neutron-ray, and baryon-ray.

In addition, the National Institute of Radiological Science (NIRS) began the radiation therapy with neutrons of high LET radiation and carbon since the 1990s. Currently, there are more than five radiation treatment center locations using carbon, in Japan. Furthermore, starting with treatment of patients with carbon ions in Helmholtzzentrum für Schwerionenforschung GmbH (GSI), Heidelberger Ionenstrahl-Therapiezentrum (HIT) center of the Heidelberg University Hospital, an exclusive center for patient treatment opened, and a number of high LET radiation (bayron) treatment centers, such as the Centro Nazionale di Adroterapia Oncologica (CNAO) in Italy or the MedAustron in Australia, has opened. Currently, even in China, such high LET radiation therapy centers are being constructed in two places, including Shanghai. Also in South Korea, the project to build these centers has been promoted.

Bayron therapy is high-tech therapy in the global market that is hugely in demand. The cumulative number of patients until December 2013 was 10,777 people, there are eight centers that have been run in four countries, and there are 13 centers that are in promotion (7 centers are in construction and six centers are on the plan). The 5-year survival rate (cure rate) of the major six cancers when using these high LET radiation therapy techniques is 22.3% higher than when using low LET radiation therapy, and the high LET radiation therapy techniques are efficient in treatment of various malignant tumors.

Gold nano-particles (GNPs) have been studied over a wide range, such as radiation therapy, drug treatment, photothermal therapy, and photodynamic therapy.

The cancer radiation therapy utilizes a mechanism in which high-energy radiation is reacted with water to produce free radicals, and the free radicals damage the DNA of a cell, resulting in death of the cell. Recently, it has been reported that pre-hydrated electrons generated by high-energy radiation also directly affect DNA.

When using nano-particles in cancer radiation therapy, absorption of radiation photons of cells may increase, resulting in generation of numerous free radicals. Further, when GNPs, a high Z material, are used, generation of 2^{nd} electron may be activated, resulting in activation of generation of free radicals. Eventually, damage to the cells deepens. Thus, GNPs are known to serve as a radiosensitizer in cancer radiation therapy.

In cancer radiation therapy, it is known that GNPs are effective as a radiosensitizer (Non-patent document 1). However, it is known that GNPs are effective as a radiosensitizer in the case of cancer treatment with low LET radiation therapy, only.

### PRIOR ART DOCUMENT

### Non-Patent Document

Reports of Practical Oncology & radiation therapy, Volume 15, Issue 6, November-December 2010, Pages 176-180

### SUMMARY

The present disclosure provides a method of enhancing neutron high linear-energy-transfer (LET) radiation therapy, which is significantly effective in radiation therapy of cancer.

Additional aspects will be set forth in part in the description which follows and, in part, will be apparent from the description, or may be learned by practice of the presented embodiments.

According to an aspect of the present disclosure, there is provided a method of enhancing neutron high LET radiation therapy of cancer, wherein the method includes administering an effective amount of gold nano-particles (GNPs) to a subject that needs neutron high LET radiation therapy of cancer, before or after radiation therapy.

According to another aspect of the present disclosure, there is provided a method of increasing sensitivity of cancer cells to neutron high LET radiation, wherein the method includes adding GNPs to the cancer cells in vitro or ex vivo and then leaving the cancer cells.

### BRIEF DESCRIPTION OF THE DRAWINGS

These and/or other aspects will become apparent and more readily appreciated from the following description of the embodiments, taken in conjunction with the accompanying drawings in which:

FIG. 1 illustrates microscope images of liver cancer cells, Huh7 and HepG2, which were treated with gold nano-particles (GNPs) labeled with a fluorescent material, Cy-5.5 for about 16 hours;

FIG. 2A is a graph illustrating results of colony formation assay staining performed on a liver cancer cell line, Huh7 or HepG2, 14 days after pretreating the liver cancer cell line with GNPs for about 16 hours and irradiating the liver cancer cell line with cesium (Cs), i.e., low LET radiation, or neutrons, i.e., high linear-energy-transfer (LET) radiation, at intensities of 2 Gy, 4 Gy, 6 Gy, and 8 Gy;

FIG. 2B is a graph illustrating results of fluorescence activated cell sorting (FACS) analysis, about a degree of apoptosis, on a liver cancer cell line, Huh7 or HepG2, 14 days after pretreating the liver cancer cell line with GNPs for about 16 hours and irradiating the liver cancer cell line with Cs, i.e., low LET radiation, or neutrons, i.e., high LET radiation, at intensities of 2 Gy, 4 Gy, 6 Gy, and 8 Gy;

FIG. 2C illustrates results of Western blot, about protein expression change of cleaved PARP-1, performed on a liver cancer cell line, Huh7 or HepG2, 14 days after pretreating the liver cancer cell line with GNPs for about 16 hours and irradiating the liver cancer cell line with Cs, i.e., low LET radiation, or neutrons, i.e., high LET radiation, at intensities of 2 Gy, 4 Gy, 6 Gy, and 8 Gy;

FIG. 3A illustrates cell cycle distribution resulting from FACS analysis, after treating a liver cancer cell line, Huh7 or HepG2, with GNPs, and after 16 hours, irradiating the liver cancer cell line with Cs, i.e., low LET radiation, or neutrons, i.e., high LET radiation, at an intensity of 5 Gy;

FIG. 3B illustrates results of protein expression change of cyclin B1, resulting from Western blot, after treating liver cancer cells, Huh7 or HepG2, with GNPs, and after 16 hours, irradiating the liver cancer cells with Cs, i.e., low LET radiation, or neutrons, i.e., high LET radiation, at an intensity of 5 Gy;

FIG. 4A illustrates images of liver cancer cells, Huh7 or HepG2, after pretreating the liver cancer cells with GNPs for about 6 hours or about 24 hours, irradiating the liver cancer cells with Cs, i.e., low LET radiation, or neutrons, i.e., high LET radiation, at an intensity of 5 Gy, and immunofluorescence-staining γ-H2AX, a DSB marker protein, with green;

FIG. 4B is a graph of expression amounts of γ-H2AX proteins in liver cancer cells, Huh7 or HepG2, after treating the liver cancer cells with GNPs for about 6 hours or about 24 hours, irradiating the liver cancer cells with Cs, i.e., low LET radiation, or neutrons, i.e., high LET radiation, at an intensity of 5 Gy, and immunofluorescence-staining γ-H2AX, a DSB marker protein, with green;

FIG. 5A illustrates images of results of wound healing assay regarding migration of liver cancer cell lines, in which liver cancer cells, Huh7 or HepG2, were pretreated with GNPs, and after 16 hours, irradiated with Cs, i.e., low LET radiation, or neutrons, i.e., high LET radiation, at an intensity of 5 Gy;

FIG. 5B is a graph illustrating results of migration ratio of the liver cancer cell lines;

FIG. 6A illustrates images of results of infiltration analysis regarding infiltration of liver cancer cell lines, in which liver cancer cells, Huh7 or HepG2, were pretreated with GNPs, and after 16 hours, irradiated with Cs, i.e., low LET radiation, or neutrons, i.e., high LET radiation, at an intensity of 5 Gy; and

FIG. 6B is a graph illustrating results of infiltration ratio of the liver cancer cell lines.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

The present invention will be described in further detail.

Unless otherwise defined, all terms (including technical and scientific terms) used herein have the same meaning as commonly understood by one of ordinary skill in the art to which this invention belongs. Although exemplary methods or materials are listed herein, other similar or equivalent ones are also within the scope of the present invention. All publications disclosed as references herein are incorporated in their entirety by reference.

An aspect of the present disclosure provides a method of enhancing neutron high linear-energy-transfer (LET) radiation therapy of cancer, wherein the method may include administering an effective amount of gold nano-particles (GNPs) to a subject that needs neutron high LET radiation therapy of cancer, before or after radiation therapy.

As used herein, the term “GNPs” may refer to particles of gold having a diameter in the nanometer scale, and include particles of gold having a diameter in the nanometer scale suitable as an effective radiosensitizer. If necessary, the surface of the GNPs may be modified. In some embodiments, a diameter of the GNPs may be in a range of about 1.9 nm to about 50.0 nm. Within this range, the GNPs may exhibit an excellent radiation sensitivity enhancing effect.

The GNPs may be prepared according to one or more suitable methods known in the art or may be purchased in the market. For example, GNPs may be prepared as follows: HAuCl₄, which is a gold source, may be reduced using sodium citrate, i.e., a reducing agent, to prepared GNPs. In this case, the size of GNPs may be adjusted by changing the amount of citrate added thereto. In other words, as the more citrate is added thereto, the more nucleation occurs, thus reducing the size of GNPs.

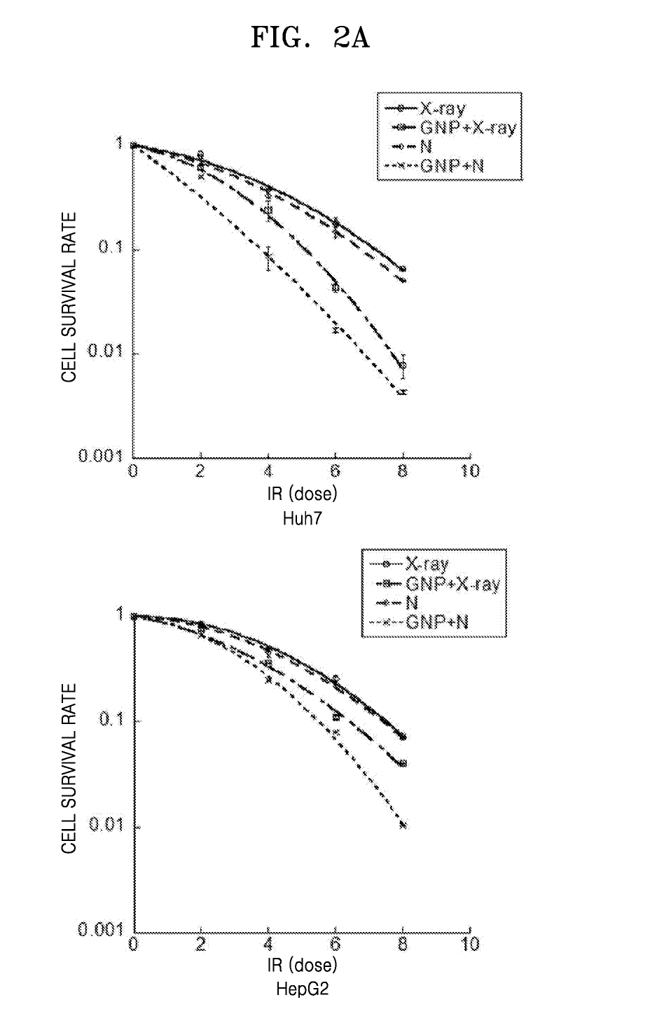

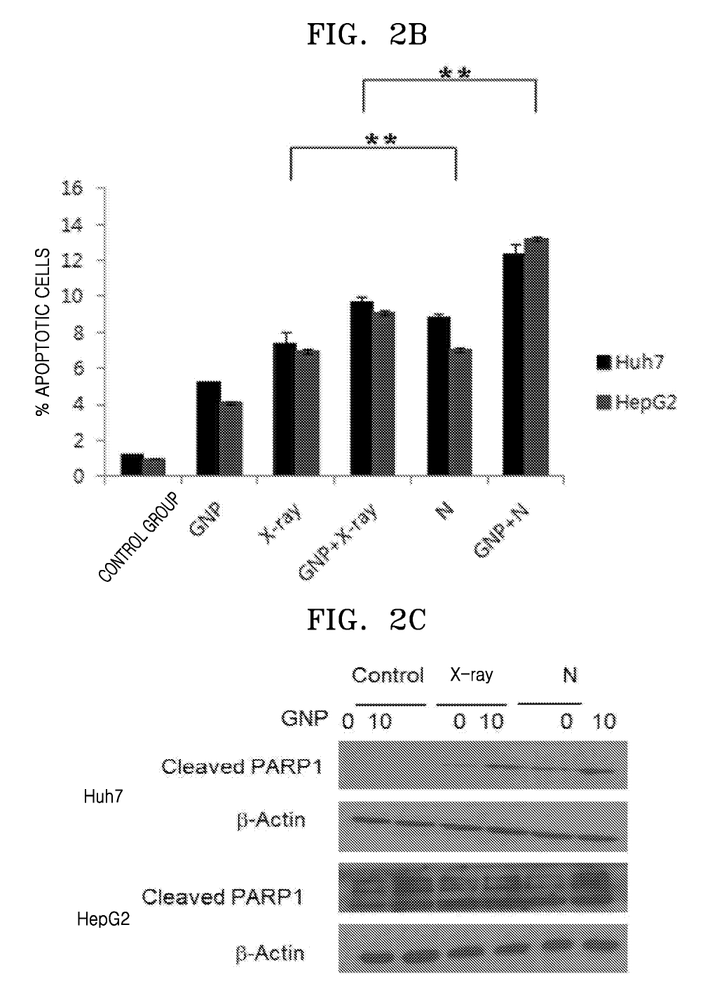

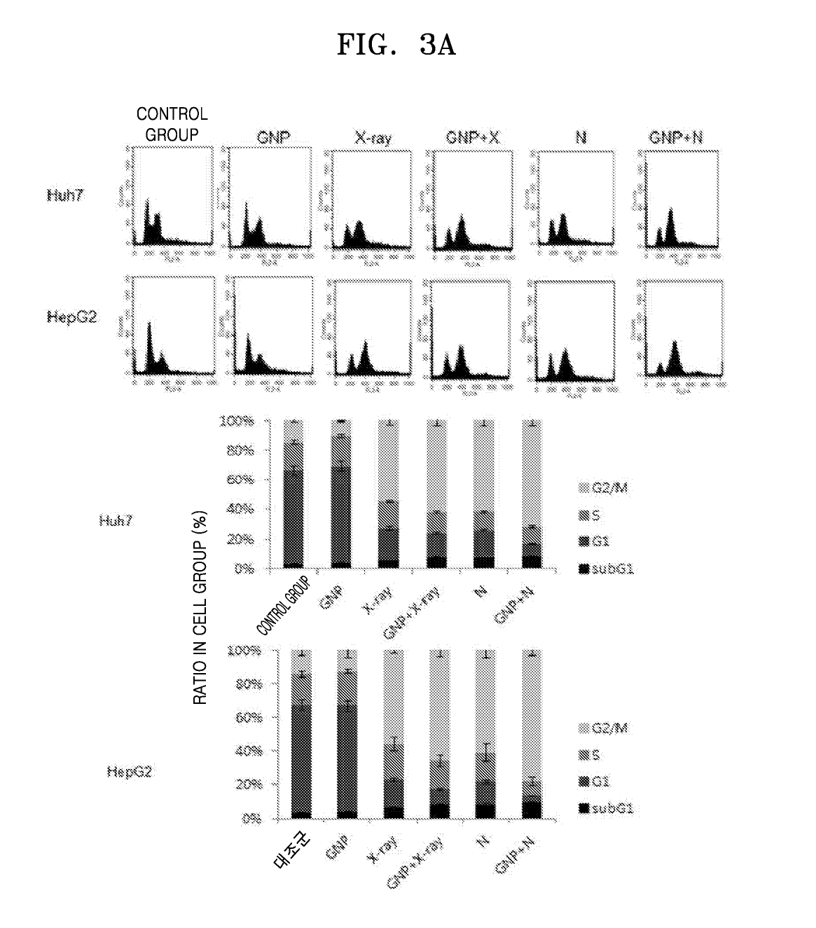

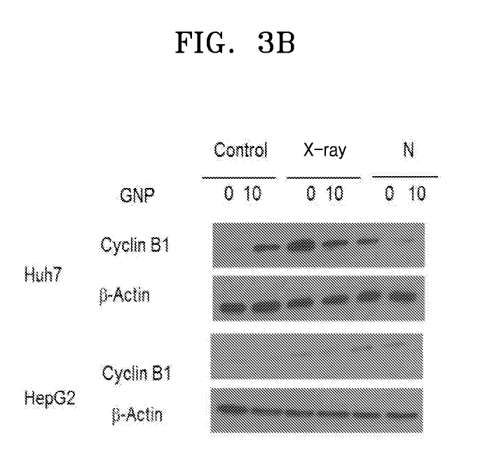

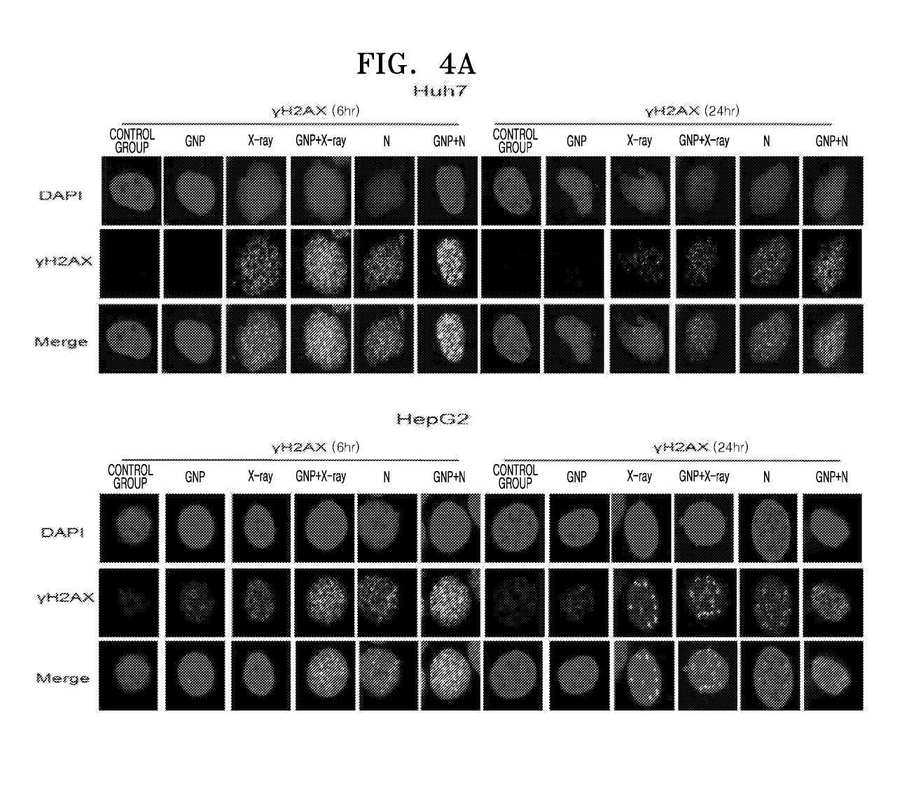

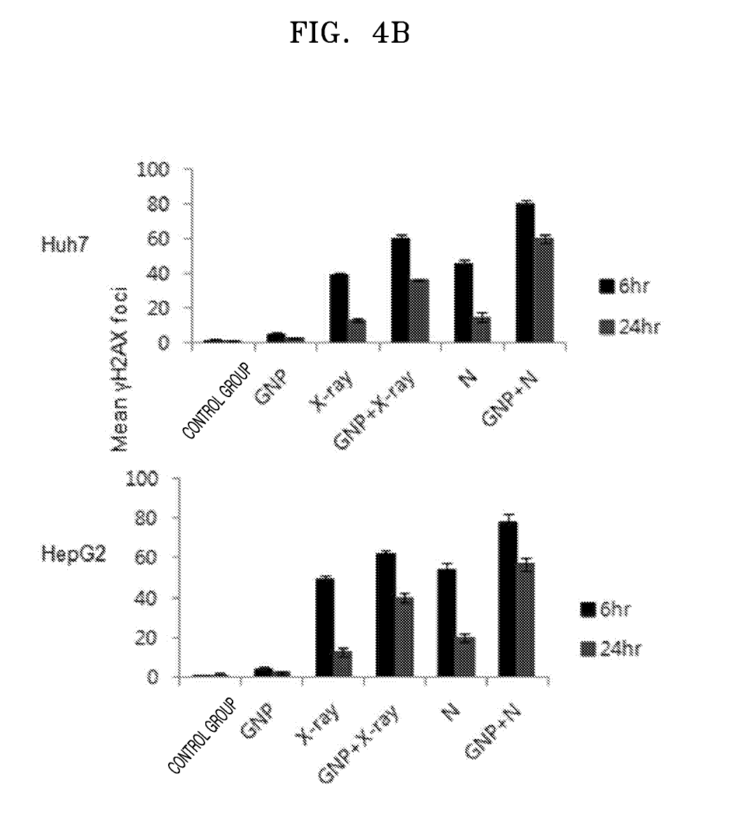

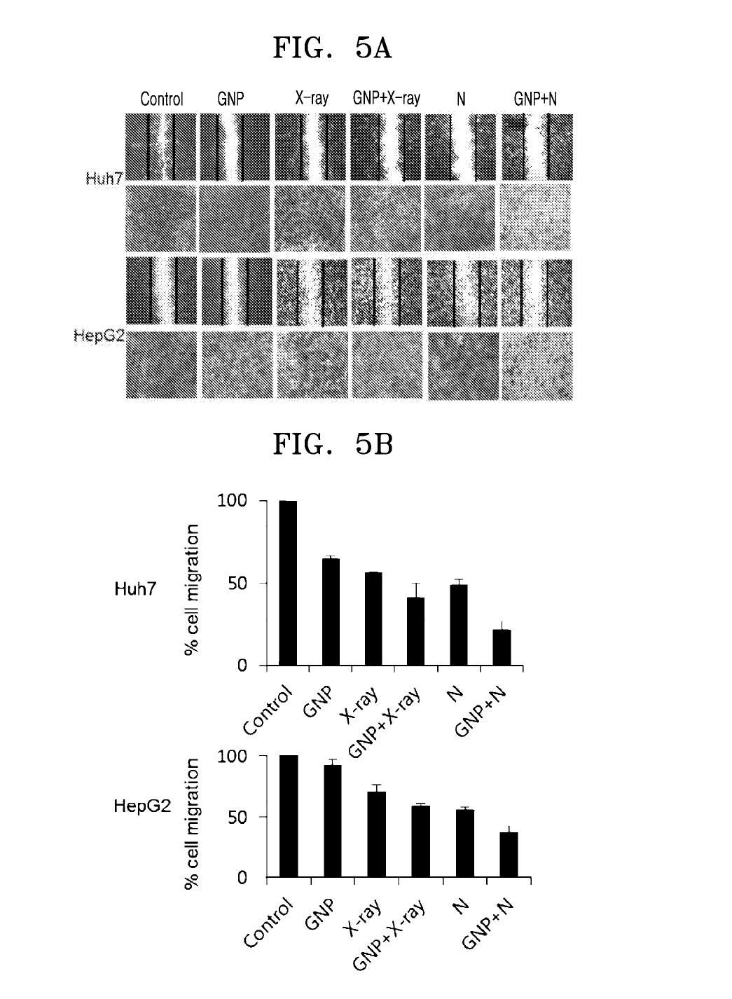

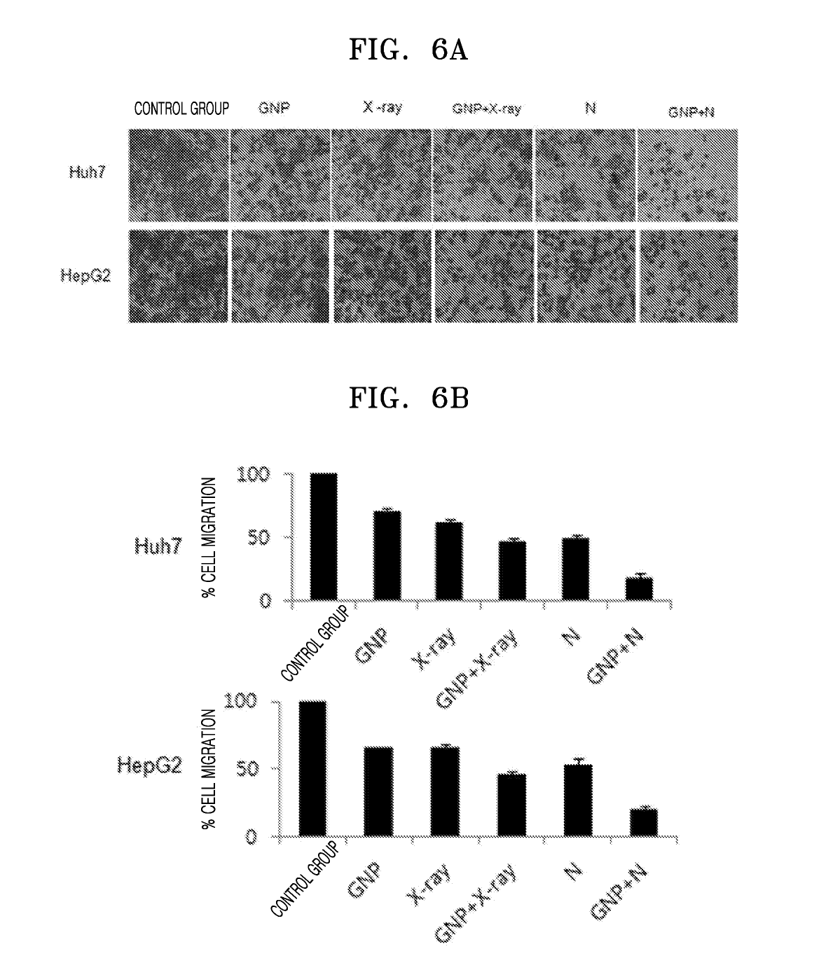

As an experimental result, a liver cancer cell line, Huh7 or HepG2, was pretreated with GNPs for about 16 hours, and then irradiated with cesium (Cs), i.e., low LET radiation, or neutrons, i.e., high LET radiation, at intensities of 2 Gy, 4 Gy, 6 Gy, and 8 Gy. After 14 days, a degree of apoptosis was analyzed by performing fluorescence activated cell sorting (FACS) analysis. As a result, it was found that a degree of increase of apoptosis of cancer cells in the case of irradiation with neutron radiation with GNPs was significantly high, as compared with that of irradiation with neutron radiation only, without GNPs (Example 2: FIGS. 2A to 2C). A liver cancer cell line, Huh7 or HepG2, was pretreated with GNPs, after 16 hours, irradiated with Cs, i.e., low LET radiation, or neutrons, i.e., high LET radiation, at an intensity of 5 Gy, and then cell cycle distribution was analyzed by performing FACS analysis. As a result, it was found that a degree of increase of G2/M arrest in the case of irradiation with neutron radiation was significantly high, as compared with that of irradiation with low LET radiation (Example 3: FIGS. 3A and 3B). Liver cancer cells, Huh7 or HepG2, were treated with GNPs, after 6 or 24 hours, irradiated with Cs, i.e., low LET radiation, or neutron, i.e., high LET radiation, at an intensity of 5 Gy, and then immunofluorescence-staining of γ-H2AX, which is for restoring damage to a DNA double helix structure and is a DSB marker protein, was carried out. As a result, it was found that the expression of γ-H2AX, in the case of irradiation with neutron radiation, was maintained continuously significantly, as compared with that of irradiation with low LET radiation, thus exhibiting that a delay degree of restoring damage to DNA was higher in the case of irradiation with neutron radiation (Example 4: FIGS. 4A and 4B). Liver cancer cells, Huh7 or HepG2, was pretreated with GNPs, after 16 hours, irradiated with Cs, i.e., low LET radiation, or neutrons, i.e., high LET radiation, at an intensity of 5 Gy, and then migration of the liver cancer cell line was analyzed by performing wound healing assay and infiltration analysis. As a result, it was found that a degree of decrease of migration and infiltration of the liver cancer cell line in the case of irradiation with neutron radiation was significantly high, as compared with that of irradiation with low LET radiation (Example 5: FIGS. 5A and 5B and FIGS. 6A and 6B). Accordingly, as proved in the foregoing experiments, it was found that when GNPs are used in conjunction with neutron radiation, i.e., high LET radiation, all aspects, including increase of apoptosis, delay of restoring damage to DNA, and prevention of metastasis in cancer cells, were excellent beyond expectation in terms of the increase of the effects, as compared with that when GNPs are used in conjunction with low LET radiation. In conclusion, when GNPs are used in conjunction with high LET radiation therapy, GNPs are excellent as radiosensitizers, and furthermore, when comparing with low LET radiation therapy, the effect as radiosensitizers is significantly excellent.

Therefore, the GNPs may be used in conjunction with high LET radiation therapy, so as to increase an anticancer effect and effect of preventing cancer metastasis, when performing high LET radiation therapy on various cancers.

The cancer may be any cancer that may be treated by performing high LET radiation therapy, and the cancer may be any one selected from the group consisting of liver cancer, lung cancer, breast cancer, prostate cancer, testicular cancer, colon cancer, stomach cancer, peritoneal cancer, kidney cancer, bladder cancer, thyroid cancer, pancreatic cancer, gallbladder cancer, biliary tract cancer, non-Hodgkin's lymphoma, lip cancer, tongue cancer, acute myelocyte leukemia, basal cell cancer, brain tumor, skin cancer, and Kaposi sarcoma, but the cancer is not limited thereto. In some embodiments, the cancer may be liver cancer.

As used herein, the term “treatment” by performing neutron high LET radiation on cancer may mean not only treating cancer by using neutron high LET radiation, but also preventing, improving, preventing aggravation, and treating morbidity of cancer patients, such as preventing the recurrence of cancer or suppressing proliferation of cancer cells.

The “treatment” by performing neutron high LET radiation on cancer may include preventing or treating metastasis of cancer. The present inventors found that, when a liver cancer cell line is pretreated with GNPs and irradiated with radiation, and the migration of the liver cancer cell line is analyzed by performing wound healing assay and infiltration analysis, a degree of decrease of migration and infiltration of the liver cancer cell line was significantly high in the case of irradiation with neutron radiation, as compared with that of irradiation with low LET radiation (Example 5: FIGS. 5A and 5B and FIGS. 6A and 6B). Therefore, GNPs are found to be highly effective when used in conjunction with radiation therapy for preventing or treating metastasis of cancer.

In some embodiments, GNPs may be administered to an adult, who weighs about 60 kilograms (kg) as a reference, at an amount of about 1 milligrams (mg) to about 100 mg, to enhance neutron radiation therapy for cancer. GNPs may be administered, before radiation treatment, while performing radiation treatment, or after radiation treatment. In an embodiment, GNPs may be administered before radiation treatment.

GNPs may be administered via any suitable administration route if the GNPs serve as radiosensitizers for cancer, and the GNPs may be combined with pharmaceutically acceptable additives to form a pharmaceutical composition. The pharmaceutical composition may be formulated into any suitable pharmaceutical dosage form known in the art, depending on an administration route.

In some embodiments, a pharmaceutical composition including the GNPs may be administered by injection, and the pharmaceutical composition may be formulated into an injection. When the pharmaceutical composition is formulated into an injectable formulation, a non-toxic buffer solution that is isotonic with blood may be used as a diluting agent. An example of the non-toxic buffer solution may be a phosphoric acid buffer solution of pH 7.4. The pharmaceutical composition may include other diluting agents or additives in addition to the buffer solution. The diluting agents or additives that may be added to the injection are known in the art, and, for example, may be known in light of the following document (Dr. H. P. Fiedler “Lexikon der Hilfsstoffe fur Pharmazie, Kosmetik and angrenzende Gebiete” [Encyclopedia of auxiliaries for pharmacy, cosmetics and related fields]).

Another aspect of the present disclosure provides a method of increasing sensitivity of cancer cells to neutron high LET radiation, wherein the method includes adding GNPs to the cancer cells in vitro or ex vivo and then leaving the cancer cells.

The description of the method of enhancing neutron high LET radiation therapy of cancer using GNPs, according to an aspect of the present disclosure, may be applied to the detailed description of the method of increasing sensitivity of cancer cells to neutron high LET radiation.

When the adding of GNPs to the cancer cells in vitro or ex vivo and then the leaving of the cancer cells is included, the sensitivity of the cancer cells to radiation, in the case of irradiation with the neutron high LET radiation, may increase. The adding of GNPs to the cancer cells in vitro or ex vivo and then the leaving of the cancer cells may be carried out before irradiating with high LET radiation, while irradiating high LET radiation, or after irradiating with high LET radiation. In some embodiments, the adding of GNPs to the cancer cells and then the leaving of the cancer cells may be carried out as pretreatment, before irradiating with high LET radiation.

In the adding of GNPs to the cancer cells and then the leaving of the cancer cells, a period of leaving the cancer cells is not particularly limited, only if the period is to increase the sensitivity of the cancer cells to neutron high LET radiation. In some embodiments, the cancer cells may be left for about 6 hours to about 24 hours to thereby sufficiently increase the sensitivity of the cancer cells.

The expression “sensitivity of cancer cells to neutron high LET radiation” may refer to increase of any reaction of cancer cells by neutron high LET radiation. In some embodiments, the sensitivity of the cancer cells to neutron high LET radiation may include sensitivity to any phenomenon selected from the group consisting of increase of apoptosis of cancer cells, increase in the number of cells in G2/M arrest in a cell cycle, decrease of cyclin-B1, continuation of an expression amount of γ-H2AX, a DSB marker protein, delay of restoring damage to DNA in cancer cells, and decrease of migration and infiltration of a cancer cell line, but the sensitivity is not limited thereto.

The cancer cells may be any cancer cell line, for example, the cancer cells may be a liver cancer cell line, a lung cancer cell line, a breast cancer cell line, a prostate cancer cell line, a testicular cancer cell line, a colon cancer cell line, a stomach cancer cell line, a peritoneal cancer cell line, a kidney cancer cell line, a bladder cancer cell line, a thyroid cancer cell line, a pancreatic cancer cell line, a gallbladder cancer cell line, a biliary tract cancer cell line, a non-Hodgkin's lymphoma cell line, a lip cancer cell line, a tongue cancer cell line, an acute myelocyte leukemia cell line, a basal cell cancer cell line, a brain tumor cell line, a skin cancer cell line, or a Kaposi sarcoma cell line, but embodiments are not limited thereto. In some embodiments, the cancer cell line may be a liver cancer cell line, Huh7 or HepG2.

As described above, according to the one or more embodiments of the present disclosure, when used with neutron high LET radiation therapy of cancer, GNPs significantly increase a radiation sensitizing effect, as compared with when used with low LET radiation therapy. Accordingly, it was found that GNPs are significantly effective in view of promoting apoptosis of cancer cells or suppressing of metastasis of cancer cells during radiation therapy. Therefore, one or more embodiments of the present disclosure provide a method of enhancing radiation therapy unexpectedly effective, in the case of neutron high LET radiation therapy of cancer.

Hereinafter, one or more embodiments of the present disclosure will be described in detail with reference to the following examples. However, these examples are not intended to limit the scope of the one or more embodiments of the present disclosure.

Experiment Method

(1) Culture of Cell Line to Use

Human liver cancer cells (Huh7 and HepG2) were purchased at the Cell Line Bank of Seoul National University, and cultured in a culture medium in an incubator maintained at 5% CO₂ and 37° C.

(2) Irradiation with Radiation

The human liver cancer cells were cultured on 3.5 cm, 6 cm, and 10 cm culture dishes in a CO₂ incubator maintained at 37° C., until the human liver cancer cells grew about 70% to about 80% confluency. Then, the human liver cancer cells were irradiated with gamma-rays from a ¹³⁷Cs gamma-ray source (available from Atomic Energy of Canada Ltd., Canada) at a dose rate of 3.81 Gy/minutes and with about 9.8 MeV neutrons (about 30 to about 40 keV/mm). If necessary, the human liver cancer cells were irradiated at a dose of 2 Gy and 5 Gy.

(3) Analysis of Proteins Using Electrophoresis and Immune Response

After the cultured cells were irradiated with radiation, in order to observe the proteins in the cultured cells, the cultured cells were dissolved in a solution consisting of 150 mM of sodium chloride, 40 mM Tris-Cl (pH 8.0), and 0.1% NP-40 to prepare a sample. This sample underwent sodium dodecyl sulfate polyacrylamide gel electrophoresis (SDS-PAGE), followed by Western blot. Electrophoretically separated proteins were transferred to nitrocellulose membranes, followed by immunoblotting to analyze an expression level of the proteins.

(4) Colony Formation Assay Staining

The cultured cells were treated with GNPs, and 12 hours after, irradiated with radiation, followed by 14 days of incubating. Then, once a colony was formed, the colony was fixed with 100% methanol and stained with 0.4% crystal violet (Sigma, St. Louis, Mo., USA) to analyze a colony formation ratio.

(5) Cell Fluorescence Staining

Human liver cancer cells were cultured on a cover slide, irradiated with radiation, fixed with 10% neutral formalin for about 10 minutes, and rinsed with phosphate buffered saline (PBS) before staining. The human liver cancer cells were permeabilized with 2% Triton X-100 and blocked in 4% FBS bovine serum albumin. γ-H2AX antibody (available from Millipore) with 1:100 dilution in a PBS (including 0.1% Triton x-100) reacted at a temperature of about 4° C. for about 24 hours. After rinsing with PBS, an antibody with a fluorescent material, as a secondary antibody, was diluted therewith at 1:500 and reacted at a temperature of about 25° C. for about 2 hours. In order to stain a nucleus, a DAPI fluorescent material was diluted with PBS at 1:1000 and reacted at a temperature of about 25° C. for about 30 minutes. Once the reaction was complete, the cells were washed with a PBS solution, and a drop of glycerol was applied thereto, followed by covering with a cover slide to observe the cells with a confocal microscope.

(6) Wound Healing Assay

The liver cancer cell line were placed on 6 well plates, scratch-wounded with a sterilized pipette tip at 90% of confluence, treated with GNPs, and 16 hours after, irradiated with radiation, followed by analysis of cell migration distance using J image.

### Example 1

### Test of Uptake of Liver Cancer Cells after Treated with GNPs

Liver cancer cells (Huh7 and HepG2) were treated with GNPs labeled with a fluorescent material, Cy-5.5, at a concentration of 10 μM, and after 16 hours, the liver cancer cells were fixed to observe them with a microscope.

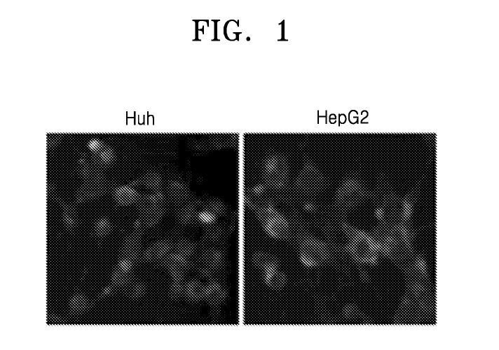

FIG. 1 illustrates microscopic images of the liver cancer cells, Huh7 and HepG2, which were treated with GNPs labeled with Cy-5.5 for about 16 hours.

Referring to FIG. 1, it was found that when the liver cancer cells, Huh7 and HepG2, were treated with GNPs, the GNPs were uptaken into the liver cancer cells.

### Example 2

### Test of Radiation Sensitivity to High LET Radiation Using GNPs

In order to identify the enhancing effect of radiation sensitivity when using radiation in conjunction with GNPs, cultured liver cancer cell lines, Huh7 and HepG2, were pretreated with GNPs for about 16 hours, and then irradiated with Cs, i.e., low LET radiation, or neutrons, i.e., high LET radiation, at intensities of 2 Gy, 4 Gy, 6 Gy, and 8 Gy. After 14 days, colony formation assay staining was performed thereon. As a result of the colony formation assay staining, a colony formation ratio was identified. When Cs or neutron were irradiated at an equivalent dose, the enhancing effect of radiation sensitivity was exhibited both in use of low LET radiation (Cs irradiation) and of high LET radiation (neutron irradiation). Particularly, the enhancing effect of radiation sensitivity in the case of irradiation with high LET radiation in conjunction with GNPs was significantly high, as compared with that of irradiation of low LET radiation in conjunction with GNPs. The results thereof are shown in FIG. 2A.

Further, separately, after the liver cancer cell lines were treated with radiation, a degree of apoptosis was analyzed by performing FACS analysis, and the protein expression change of cleaved PARP-1, which is known as a marker protein of apoptosis, was tested by performing Western blot. The results thereof are shown in FIGS. 2B and 2C.

Referring to FIG. 2B, as a result of FACS analysis, to test a degree of apoptosis, on the two liver cancer cells irradiated with low LET radiation and high LET radiation in conjunction with GNPs, the enhancing effect of radiation sensitivity was exhibited both in use of low LET radiation (Cs irradiation) and of high LET radiation (neutron irradiation). Particularly, the enhancing effect of radiation sensitivity in the case of treatment with high LET radiation in conjunction with GNPs was significantly high, as compared with that of treatment of low LET radiation in conjunction with GNPs.

Referring to FIG. 2C, it was found that the enhancement of the expression rate of the cleaved PARP-1 was exhibited both in use of low LET radiation (Cs irradiation) and of high LET radiation (neutron irradiation). Particularly, the enhancement of the expression rate of the cleaved PARP-1 in the case of treatment with high LET radiation in conjunction with GNPs was significantly high, as compared with that of treatment of low LET radiation in conjunction with GNPs.

### Example 3

### Test of Cell Cycle by FACS Analysis for Liver Cancer Cell Line Treated with High LET Radiation and Low LET Radiation

In order to test cell cycle distribution in the case of treatment with high LET radiation and with low LET radiation in conjunction with GNPs, cultured liver cancer cell lines, Huh7 and HepG2, were pretreated with GNPs for about 16 hours, irradiated with Cs, i.e., low LET radiation, or neutrons, i.e., high LET radiation, at an intensity of 5 Gy, and then, after 24 hours, fixed with 70% ethanol. FACS analysis was performed thereon, and the results thereof are shown in FIG. 3A. In addition, protein expression change of cyclin B1, which is a protein that regulates G2/M phase of a cell cycle, was tested by using Western blot. The results thereof are shown in FIG. 3B.

FIG. 3A illustrates cell cycle distribution resulting from FACS analysis, after treating the liver cancer cells, Huh7 or HepG2, with GNPs, and after 16 hours, irradiating the liver cancer cells with Cs, i.e., low LET radiation, or neutrons, i.e., high LET radiation, at an intensity of 5 Gy.

FIG. 3B illustrates the results of the protein expression change of cyclin B1, resulting from Western blot, after treating the liver cancer cells, Huh7 or HepG2, with GNPs, and after 16 hours, irradiating the liver cancer cells with Cs, i.e., low LET radiation, or neutrons, i.e., high LET radiation, at an intensity of 5 Gy.

Referring to the results of FIG. 3A, it was found that treatment with high LET radiation and with low LET radiation induced G2/M arrest, and further, in the case of irradiation in conjunction with GNPs, G2/M arrest increased. Particularly, the increase degree of G2/M arrest in the case of treatment with high LET radiation in conjunction with GNPs was significantly high, as compared with that of treatment with low LET radiation in conjunction with GNPs.

Referring to the results of FIG. 3B, it was found that cyclin B1 decreased in the cases of treatment with high LET radiation only and of treatment with low LET radiation only, as compared with that of the control group, and further, in the case of irradiation in conjunction with GNPs, cyclin B1 decreased to a greater degree. Particularly, the decrease degree of cyclin B1 in the case of treatment with high LET radiation in conjunction with GNPs was significantly high, as compared with that of treatment with low LET radiation in conjunction with GNPs.

### Example 4

### Test of Expression Amount of DNA Restoring Proteins in Liver Cancer Cells in the Case of High LET Radiation in Conjunction with GNPs, by Using Cell Fluorescent Staining

In order to observe an expression amount of a DSB marker protein which is for restoring damage to a DNA double helix structure, caused by irradiation with radiation, liver cancer cell lines, Huh7 or HepG2, were pretreated with GNPs for about 6 hours or about 24 hours, and then irradiated with high LET radiation or low LET radiation. γ-H2AX, which is a DSB marker protein, was immunofluorescence-stained with green color. The expression of γ-H2AX, which is a marker for damage to a DNA double helix structure, may refer to a degree of damage to a DNA double helix structure in the case of irradiation with radiation. As a control experiment, a nucleus was stained with DAPI, a blue fluorescent material. The results thereof are shown in FIGS. 4A and 4B.

FIG. 4A illustrates images of the liver cancer cells, Huh7 or HepG2, after pretreating the liver cancer cells with GNPs for about 6 hours or about 24 hours, irradiating the liver cancer cells with Cs, i.e., low LET radiation, or neutrons, i.e., high LET radiation, at an intensity of 5 Gy, and immunofluorescence-staining γ-H2AX, a DSB marker protein, with green.

FIG. 4B is a graph of expression amounts of γ-H2AX proteins in the liver cancer cells, Huh7 or HepG2, after treating the liver cancer cells with GNPs for about 6 hours or about 24 hours, irradiating the liver cancer cells with Cs, i.e., low LET radiation, or neutrons, i.e., high LET radiation, at an intensity of 5 Gy, and immunofluorescence-staining γ-H2AX, a DSB marker protein, with green.

Referring to FIGS. 4A and 4B, there is little damage to DNA in the control group. 24 hours after treating with low LET radiation only and with high LET radiation only, the expression amounts of γ-H2AX decreased, indicating that damage to DNA was being restored. Whereas, when treated with radiation in conjunction with GNPs, it was found that γ-H2AX proteins were still expressed.

In addition, when comparing damage restoration of the group in the case of treatment of high LET radiation in conjunction with GNPs with that of treatment of low LET radiation in conjunction with GNPs, it was found that the expression amount of the γ-H2AX proteins in the group irradiated with high LET radiation was continuously maintained, indicating that damage restoration in the group in the case of treatment of high LET radiation in conjunction with GNPs, was delayed.

### Example 5

### Observation of Cancer Metastasis Prevention in Liver Cancer Cell Line Due to High LET Radiation

In order to observe cancer metastasis prevention due to treatment of high LET radiation in conjunction with GNPs, liver cancer cell lines were pretreated with GNPs for about 16 hours, and irradiated with high LET radiation or low LET radiation at an intensity of about 5 Gy. Over time, migration and infiltration of the liver cancer cell lines had been observed by using wound healing assay and infiltration analysis. The results thereof are shown in FIGS. 5A and 5B and FIGS. 6A and 6B.

FIG. 5A illustrates images of results of wound healing assay regarding migration of liver cancer cell lines, in which liver cancer cells, Huh7 or HepG2, were pretreated with GNPs, and after 16 hours, irradiated with Cs, i.e., low LET radiation, or neutrons, i.e., high LET radiation, at an intensity of 5 Gy. FIG. 5B is a graph illustrating results of migration ratio of the liver cancer cell lines.

FIG. 6A illustrates images of results of infiltration analysis regarding infiltration of liver cancer cell lines, in which liver cancer cells, Huh7 or HepG2, were pretreated with GNPs, and after 16 hours, irradiated with Cs, i.e., low LET radiation, or neutrons, i.e., high LET radiation, at an intensity of 5 Gy. FIG. 6B is a graph illustrating results of infiltration ratio of the liver cancer cell lines.

Referring to FIGS. 5A and 5B and FIGS. 6A and 6B, it was found that, after the pretreatment with GNPs and irradiation with high LET radiation on the liver cancer cell lines, migration and infiltration of the cells were significantly decreased over time, as compared with those of the group irradiated with low LET radiation in conjunction with GNPs.

As a result, it was found that, when high LET radiation is used in conjunction with GNPs, radiation sensitivity increases and a cancer metastasis prevention effect also significantly increases.

While this invention has been particularly shown and described with reference to preferred embodiments thereof, it will be understood by those skilled in the art that various changes in form and details may be made therein without departing from the spirit and scope of the invention as defined by the appended claims. The disclosed embodiments should be considered in descriptive sense only and not for purposes of limitation. Therefore, the scope of the invention is defined not by the detailed description of the invention but by the appended claims, and all differences within the scope will be construed as being included in the present invention.

What is claimed is:

1. A method of enhancing neutron high linear-energy-transfer (LET) radiation therapy of cancer, wherein the method comprises administering an effective amount of gold nano-particles (GNPs) to a subject that needs high LET radiation therapy of cancer, before or after radiation therapy.

2. The method of claim 1, wherein a diameter of the GNPs is in a range of about 1.9 nm to about 50.0 nm.

3. The method of claim 1, wherein the cancer is liver cancer, lung cancer, breast cancer, prostate cancer, testicular cancer, colon cancer, stomach cancer, peritoneal cancer, kidney cancer, bladder cancer, thyroid cancer, pancreatic cancer, gallbladder cancer, biliary tract cancer, non-Hodgkin's lymphoma, lip cancer, tongue cancer, acute myelocyte leukemia, basal cell cancer, brain tumor, skin cancer, or Kaposi sarcoma.

4. The method of claim 1, the method being for preventing or treating metastasis of cancer.

5. The method of claim 1, the GNPs are administered as an injection.

6. A method of increasing sensitivity of cancer cells to neutron high linear-energy-transfer (LET) radiation, wherein the method comprises adding gold nano-particles (GNPs) to the cancer cells in vitro or ex vivo and then leaving the cancer cells.

7. The method of claim 6, wherein a diameter of the GNPs is in a range of about 1.9 nm to about 50.0 nm.

8. The method of claim 6, wherein the cancer cells are liver cancer cells, lung cancer cells, breast cancer cells, prostate cancer cells, testicular cancer cells, colon cancer cells, stomach cancer cells, peritoneal cancer cells, kidney cancer cells, bladder cancer cells, thyroid cancer cells, pancreatic cancer cells, gallbladder cancer cells, biliary tract cancer cells, non-Hodgkin's lymphoma cells, lip cancer cells, tongue cancer cells, acute myelocyte leukemia cells, basal cell cancer cells, brain tumor cells, skin cancer cells, or Kaposi sarcoma cells.

9. The method of claim 6, wherein the method is for decreasing migration or infiltration of cancer cells.
